(19) United States
(12) Patent Application Publication
(10) Pub. No.: US 2019/0150823 A1
(43) Pub. Date: May 23, 2019

(54) ADAPTIVE SUPPORT DEVICE AND SYSTEM RESPONSIVE TO CHANGING COGNITIVE ABILITY

(71) Applicant: EMMETROS LIMITED, Fergus (CA)

(72) Inventors: Mary Pat HINTON, Fergus (CA); Jennifer Lynn KRUL, Elora (CA); Patricia Lynne COOPER BARFOOT, Waterloo (CA); Alexander John DLUGOKECKI, Kitchener (CA)

(21) Appl. No.: 16/198,835

(22) Filed: Nov. 22, 2018

Related U.S. Application Data
(60) Provisional application No. 62/590,019, filed on Nov. 22, 2017.
(60) Provisional application No. 62/632,462, filed on Feb. 20, 2018.

(51) Int. Cl. A61B 5/00; G06F 9/451

(52) U.S. Cl. CPC A61B 5/4088; G06F 9/451; A61B 5/4842; G06N 20/00; A61B 5/7435; A61B 5/7267; A61B 5/7475

(57) ABSTRACT

Systems and methods for supporting and adapting to changes in cognitive ability are provided. The system includes a user interface and a processor system, the processor system being configured to detect changes in cognitive ability based on detected interactions with the user interface. The system is configured to change its interaction with the individual based on detected changes in cognitive ability. The system is configured to change system access controls for third parties based on detected changes in cognitive ability in a manner that respects the individual.

### RELATED APPLICATIONS

This application claims priority to and the benefit of: (1) U.S. Application No. 62/590,019 filed Nov. 22, 2017, for ADAPTIVE SUPPORT DEVICE AND SYSTEM RESPONSIVE TO CHANGING COGNITIVE ABILITIES; and (2) U.S. Application No. 62/632,462 filed Feb. 20, 2018, for ADAPTIVE SUPPORT DEVICE AND SYSTEM RESPONSIVE TO CHANGING COGNITIVE ABILITY. The contents of these applications are incorporated herein by reference.

### FIELD

The present disclosure relates generally to support devices, and more specifically, to adaptive support devices and systems that are responsive to changing cognitive ability of an individual.

### BACKGROUND

Cognitive changes, including an improvement in cognitive ability or a decline in cognitive ability, may affect numerous individuals.

For example, the number of individuals who experience cognitive decline due to dementia is growing rapidly in the context of an aging world population. More generally, an individual's cognitive ability may decline over time due to other conditions such as Down syndrome, Huntington's disease, chronic traumatic encephalopathy, and traumatic brain injury.

Further, individuals may experience an improvement in cognitive ability due to recovery from injury or illness, for example during recovery from traumatic brain injury.

Detecting and adapting to changes in cognitive ability is important for the daily function of an individual experiencing cognitive changes. Technology for detecting and adapting to cognitive change would be beneficial for promoting independence and supporting an individual experiencing cognitive change.

Accordingly, there is a need for technology to support and adapt to an individual as cognitive ability changes over time.

### SUMMARY

According to a first example aspect, a method for responsively adapting a user experience provided by an electronic device is described. The method includes receiving data about a first set of user interactions with the electronic device, receiving data about a further set of user interactions with the electronic device, detecting a change in the user's cognitive ability based on the data for the first set of user interactions and the data for the further set of user interactions; and adapting the user experience provided by the electronic device in response to the detected change.

In example embodiments, the first set of user interactions and further set of user interactions with the electronic device are both performed for a purpose other than only to detect the change in the user's cognitive ability. In some examples, the first set of user interactions and further set of user interactions are interactions that occur through one or more of: an event scheduling user interface; a user interface for accessing stored photos of the user; an electronic messaging user interface; and a user information user interface.

In example embodiments, the method is performed at one or more servers that communicate with the electronic device through a communication network. In some examples, detecting a threshold change in a quantity of interactions occurring in the further set of user interactions compared to the first set of user interactions. In some examples, detecting the change in the user's cognitive ability comprises detecting a threshold change in one or more of: a number of complex words input in the further set of user interactions compared to the first set of user interactions; word specificity occurring in the further set of user interactions compared to the first set of user interactions; a number of repeated words occurring in the further set of user interactions compared to the first set of user interactions; spelling accuracy of words input in the further set of user interactions compared to the first set of user interactions; a number of word classes included the further set of user interactions compared to the first set of user interactions; syntactic complexity included in user inputs in the further set of user interactions compared to the first set of user interactions; and text sentiment of user inputs in the further set of user interactions compared to the first set of user interactions.

According to a second example aspect, a system is described that includes a processor, and a memory coupled to the processor. The memory stores executable instructions that, when executed by the processor, cause the system to: receive data about a first set of user interactions with an electronic device; receive data about a further set of user interactions with the electronic device; detect a change in the user's cognitive ability based on the data for the first set of user interactions and the data for the further set of user interactions; and adapt a user experience in response to the detected change.

According to a third example aspect, a method for responsively adapting a user experience provided by an electronic device is disclosed that includes receiving data about user interactions with the electronic device; and adapting the user experience provided by the electronic device in response to detecting, based on the data about user interactions, that a user's cognitive ability is at a level that does not correspond to the user experience.

In some examples, a method for responsively adapting a system according to an adjustment in a user's cognitive ability is provided comprising: receiving a first plurality of inputs indicating user cognitive ability at a first defined level; receiving a second plurality of inputs indicating user cognitive ability at a second defined level; detecting a change in cognitive ability according to a detected difference in the first plurality of inputs compared to the second plurality of inputs; and adaptively modifying the system in response to the detected change in cognitive ability.

In some examples, the first defined level is greater than the second defined level.

In some examples, the first defined level is less than the second defined level.

In some examples, the change comprises a decrease or increase in vocabulary size in the second plurality of inputs compared to the first plurality of inputs.

In some examples, the change comprises a decrease or increase in word specificity of the second plurality of inputs compared to the first plurality of inputs.

In some examples, the change comprises an increase or decrease in the use of fillers and/or mispronunciations in the second plurality of inputs compared to the first plurality of inputs.

In some examples, the change comprises an increase or decrease in occurrence of repeated words and/or phrases in the second plurality of inputs compared to the first plurality of inputs.

In some examples, the change comprises a decrease or increase in proportion of word classes in the second plurality of inputs compared to the first plurality of inputs.

In some examples, the change comprises a decrease or increase in spelling accuracy in the second plurality of inputs compared to the first plurality of inputs.

In some examples, the change comprises a decrease or increase in syntactic complexity in the second plurality of inputs compared to the first plurality of inputs.

In some examples, the change comprises a change in text sentiment in the second plurality of inputs compared to the first plurality of inputs.

In some examples, the change comprises a decrease or increase in appropriate diction in the second plurality of inputs compared to the first plurality of inputs.

In some examples, the change comprises a decrease or increase in appropriateness of word order in the second plurality of inputs compared to the first plurality of inputs.

In some examples, the change comprises a decrease or increase in appropriateness of response to solicited input in the second plurality of inputs compared to the first plurality of inputs.

In some examples, adaptively modifying the system comprises a change to at least one of the following: image sizes, font sizes, content complexity, white space around elements and/or text, size and visual affordance of interface elements, size of touch targets, number of elements on a screen, presentation of icons and images, abstractness of images, navigation bar size and availability, availability of delete/edit functions, availability of audio output, availability and nature of support options, availability of features, third-party access and/or authority over individual's content.

In some examples, the method further comprises providing information to a user for input of a plan for the adaptive modification.

In some examples, the method further comprises passively collecting data during user input as a user interacts with the system.

In some examples, there is provided a non-transitory machine-readable medium having tangibly stored thereon executable instructions for execution by a processor of a server that, when executed by the processor, cause the server to: receive a first plurality of inputs indicating user cognitive ability at a first defined level; receive a second plurality of inputs indicating user cognitive ability at a second defined level; detect a change in cognitive ability according to a detected difference in the first plurality of inputs compared to the second plurality of inputs; and adaptively modify the system in response to the detected change in cognitive ability.

In some examples, a computer system is provided, comprising: a processor; a memory coupled to the processor, the memory storing executable instructions that, when executed by the processor, cause the server to: receive a first plurality of inputs indicating user cognitive ability at a first defined level; receive a second plurality of inputs indicating user cognitive ability at a second defined level; detect a change in cognitive ability according to a detected difference in the first plurality of inputs compared to the second plurality of inputs; and adaptively modify the system in response to the detected change in cognitive ability.

In example aspects, systems and methods are described for supporting and adapting to changes in cognitive ability are provided. The system includes a user interface and a processor system, the processor system being configured to detect changes in cognitive ability based on detected interactions with the user interface. The system is configured to change its interaction with the individual based on detected changes in cognitive ability. The system is configured to change system access controls for third parties based on detected changes in cognitive ability in a manner that respects the individual.

### BRIEF DESCRIPTION OF THE DRAWINGS

Reference is made, by way of example, to the accompanying drawings which show example embodiments of the present application, and in which:

FIG. 1 is a block diagram of a user support system that includes a user electronic device and third-party electronic device interacting with a cognitive adaption system that is enabled to detect and react to changes in cognitive ability of an individual user, according to an example embodiment;

FIG. 2 is a flow chart of an example method of adapting an individual's experience with a user device, including selection of a baseline solution and choosing accessibility customizations in accordance with an embodiment of the present disclosure;

FIG. 3 is a flowchart of an embodiment of a method for modifying elements of an individual's experience;

FIG. 4 is a block diagram of distributed network and system of an example embodiment;

FIG. 5A is an example user interface of a “My Day” home screen that may be displayed to a user with mild cognitive impairment;

FIG. 5B is an example user interface of a “My Day” home screen that may be displayed to a user with moderate cognitive impairment;

FIG. 6A is an example user interface of a “My Life” home screen that may be displayed to a user with mild cognitive impairment;

FIG. 6B is an example user interface of a “My Life” home screen that may be displayed to a user with moderate cognitive impairment;

FIG. 7A is an example user interface of a “photo album” screen that may be displayed to a user with mild cognitive impairment;

FIG. 7B is an example user interface of a “photo album” screen that may be displayed to a user with moderate cognitive impairment;

FIG. 8A is an example user interface of a “My Health” home screen that may be displayed to a user with mild cognitive impairment;

FIG. 8B is an example user interface of a “My Health” home screen that may be displayed to a user with moderate cognitive impairment;

FIG. 9A is an example user interface of a “Care provider” screen that may be displayed to a user with mild cognitive impairment;

FIG. 9B is an example user interface of a “Care provider” screen that may be displayed to a user with moderate cognitive impairment;

FIG. 10 is an example user interface of a main page screen that may be displayed to a user with severe cognitive impairment; and

FIG. 11 is an example user interface of a “Chat” screen that may be displayed to a user with mild cognitive impairment.

Similar reference numerals may have been used in different figures to denote similar components. While aspects of the present disclosure will be described in conjunction with the illustrated embodiments, it will be understood that it is not intended to limit the present disclosure to such embodiments.

### DESCRIPTION OF EXAMPLE EMBODIMENTS

The present disclosure describes systems and methods that, in various example embodiments, can detect changes in cognitive ability of an individual user, adapt the individual's technology to changes in cognitive ability, and support an individual user as cognitive ability changes by providing third-party access to at least a portion of an individual's technology. Change in cognitive ability may refer to either an improvement or a decline in cognitive ability. In example embodiments, “individual's technology” includes the experience of the individual user as the user interacts with a user electronic device, including for example aspects of a user interface presented to the individual user on the user electronic device. These aspects can include, but are not limited to: size and type of font, amount and type of information presented to the individual user, format of information, number of options presented to user, types of input and output (e.g. graphical, audible, tactile), and type of information available for access by the user. The individual's technology can include a baseline feature set, and user interface and accessibility customizations for an individual user of user support system 90. In example embodiments, “individual's technology” also extends to the experience of third party care givers (for example family members and other care providers) of the individual as they interact with aspects of a user support system relevant to the individual.

### System Environment

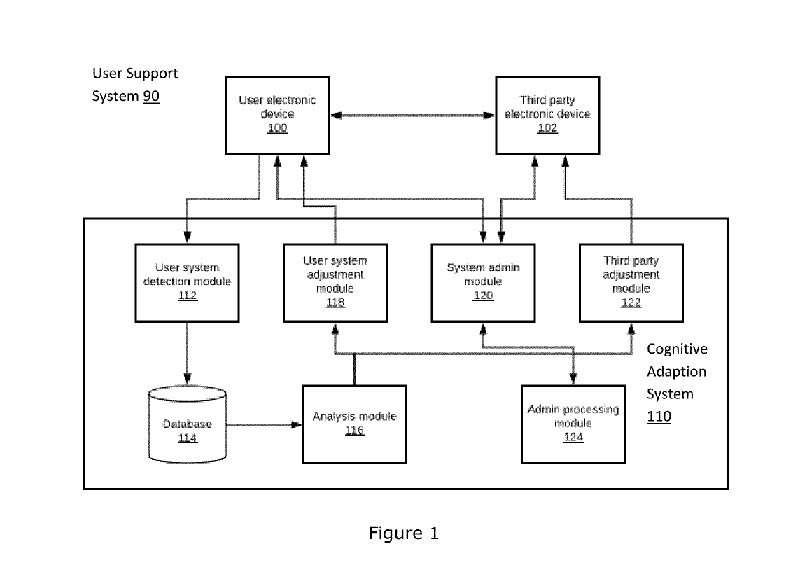

A possible environment in which example embodiments of a system for detecting and adapting to changes in cognitive ability is disclosed in FIG. 1.

With reference to FIG. 1, in example embodiments a user support system 90 is provided that includes a cognitive ability system 110 interacting with a user electronic device 100 and possibly a third-party electronic device 102. Each of user electronic device 100 and third party electronic device 102 may include, for example, a mobile apparatus, a mobile phone, a mobile communication device, a mobile computer, a laptop computer, or a desktop computer.

In at least some examples, cognitive ability system 110 is configured to detect non-intrusively how an individual user interacts with user support system 90 through user electronic device 100, and then adapt the individual's technology (for example, the user's experience with user support system 90) based on detected changes in the user's cognitive ability. User electronic device 100 and third-party electronic device 102 may include input/output (I/O) components. I/O components may include user input interfaces such as touch screens and buttons, user output interfaces such as display screens and speakers, communications interfaces for exchanging messages and data with a network and one or both of audio and video sensors such as microphones and image capturing cameras.

As shown in FIG. 1, cognitive adaption system 110 includes a plurality of modules. In the example shown in FIG. 1, the modules of cognitive ability system 110 are remotely hosted at one or more servers that communicate through one or more networks with user electronic device 100 and third-party electronic device 102. However, in various example embodiments, some of the modules include client side and server side application components that are distributed between user electronic device 100, third party electronic device 102, and one or more remote servers, and in some examples, some modules may be implemented entirely at user electronic device 100 or third party electronic device 102.

Cognitive adaption system 110 includes a user system detection module 112 for detecting input from user electronic device 100. User system detection module 112 may write and store data about user interactions with the care management system 90 through user electronic device 100 to one or more non-transitory memory resources, for example, one or more databases 114. One or more databases 114 may further include, for example, one or more different types of electronic storage elements, hard-drives and database systems, which may further be operable to store, for example, user data and program instructions that configure a processor to perform functions described herein.

Cognitive adaption system 110 may further include an analysis module 116 that may read from one or more databases 114 and perform analysis to detect a change in a user's cognitive ability. Analysis module 116 may provide output to a user system adjustment module 118 and a third-party adjustment module 122. Adjustment modules 118 and 122 may make appropriate changes to an individual's technology (including for example changes to the user's experience when interacting with user electronic device 100) and a third-party experience provided through third-party electronic device 102, as described in further detail below. Cognitive adaption system 110 may further include a system administration module 120 which interacts with user electronic device 100, third-party electronic device 102, and an administration processing module 124. A system administration module 120 may control access to content available to user electronic device 100 and third-party electronic device 102. Administration processing module 124 may interact with third-party payment systems, for example.

### Example System Network

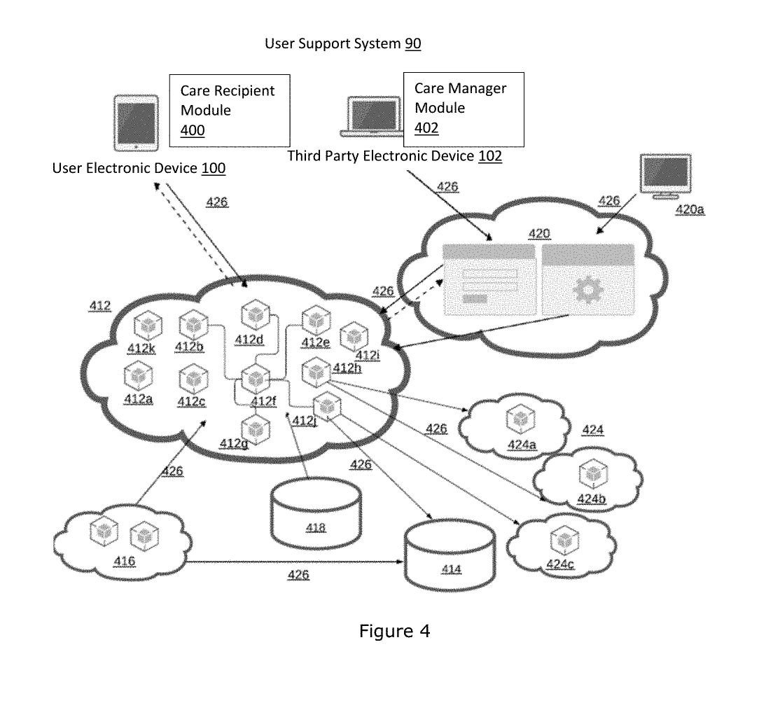

FIG. 4 is a block diagram that illustrates a distributed computer system or network upon which examples described herein may be implemented. For example, in the context of FIG. 1, the user support system 90 may be implemented using a computer network such as that described by FIG. 4.

With reference to FIG. 4, user electronic device 100 may be a processor equipped device enabled by software stored in memory of the device to implement a care recipient module 400. For example, the care recipient module 400 could be enabled by client-side software. In some examples, care recipient module 400 may include a browser user interface application for a web service that hosts user support system 90, and in some examples, a simple connection through a web browser on user electronic device 100 may be used in place of care recipient module 400. User electronic device 100 includes communications interfaces for exchanging messages and data with network 426. Further, third party electronic device 102 may be a processor equipped device enabled by software stored in memory of the device to implement a care manager module 402. For example, care manager module 402 could be enabled by client-side software. Third-party electronic device 102 includes communications interfaces for exchanging messages and data with network 426.

In example embodiments, user support system 90 includes a process/control unit 412 that is configured to detect input from, and exchange information with, user electronic device 100 and/or third-party electronic device 102. Process/control unit 412 may be hosted remotely on a single server or multiple servers and may include, as examples: a job processing module 412a which executes series of instructions; a health module 412b which processes health data to/from user electronic device 100 and/or third party device 102; a life module 412c which processes life data to/from user electronic device 100 and/or third party device 102; a day module 412d which processes day data to/from care recipient module 400 of user electronic device 100 and/or care manager module 402 of third party device 102; a chat module 412e which processes chat data to/from user electronic device 100 and/or third party device 102; an accessibility customizations module 412f which processes instructions to/from user electronic device 100 and/or third party device 102 to enhance user accessibility; an account module 412g which processes user account information to/from user electronic device 100 and/or third party device 102; an authorization module 412h which processes user access information to the other modules; a subscription module 412i which processes user payment information to the authorization module 412h; a UX orchestrator module 412j which processes the I/O interface adaptations and then directs the I/O interface on user device 100 and/or third party device 102 to respond to these adaptations; and an analytics engine 412k which processes data and provides instructions.

Process/control unit 412 may be configured to interact with one or more databases 414. One or more databases 414 may further include, for example, one or more different types of electronic storage elements, hard-drives, cloud storage systems, distributed digital ledgers, and database systems, which may further be operable to store, for example, user data and program instructions that configure a processor to operate to perform functions described herein. The Process/control unit 412 may be configured to interact with one or more electronic health record systems 418. One or more electronic health record systems 418 may further include, for example a data source for a patient-level electronic health record as well as an integration node that authorizes and executes transmission of the data.

Process/control unit 412 may further be configured to interact with hosted services 416 which may execute complex data services and artificial intelligence analysis, and third-party services, 424 which may execute a payment processing module 424a, a data reporting module 424b, and a financial reporting module 424c. Additionally, websites and clients 420 for system administration may interact with third-party electronic device 402 via a web browser or a client application. An application vendor web browser or application enabled device 420a may allow remote communication between the application vendor support team and the user.

User electronic device 100, third-party electronic device 102, and application vendor web client device 420a may communicate through communications network 426. Cloud-based services 416 and 424 and data repositories 414 and 418 may be implemented by one or more local or remotely hosted servers that communicate with user electronic device 100, third-party electronic device 102, and an application vendor web browser or application 420a through communications network 426. Communications network 426 may include one or more wired communications networks or wireless communications networks or a combination of wired and wireless communications networks. Communications network 426 may include, among other things, one or more of the internet, intranets operated by organizations or individuals, wired and wireless local area networks, wide area wireless networks such as cellular networks, cable networks, PICO networks and public switched networks.

In example embodiments, some or all or the modules of cognitive adaption system 110 are hosted on the same server or servers as process/control unit 412. In example embodiments, at least some of the modules of cognitive adaption system 110 are configured to interact with, or are integrated with, modules of the process/control unit 412. For example: user system detection module 112 is configured to interact with one or more of the modules of process/control unit 412 in order to gather individual user interaction data; user system adjustment module 112 is configured to interact with one or more of the modules of process/control unit 412 in order to change the individual's experience in interacting with user support system 90; and third party adjustment module 122 is configured to interact with one or more of the modules of process/control unit 412 in order to change the third party settings and authorizations for interacting with user support system 90.

Components of user electronic device 100, third-party electronic device 102, and cognitive adaption system 110 may interact through an ongoing or continuous process that involves the individual with cognitive impairment and one or more of their care manager, family members and/or professional care partners. In example embodiments, cognitive adaption system 110 selects an appropriate, accessible, and adapted set of features and user interface elements used by user support system 90 (including the modules of process/control unit 412) for the particular changing needs of the individual.

### Overview of System Workflow

The above description provides an example of a possible operating environment of user support system 90 system that includes cognitive adaption system 110 to detect change in an individual's cognitive ability and adapt the individual user's technology. Such an overview having been provided, an example of a possible workflow for the system to detect change in an individual's cognitive ability and adapt the individual user's technology is provided.

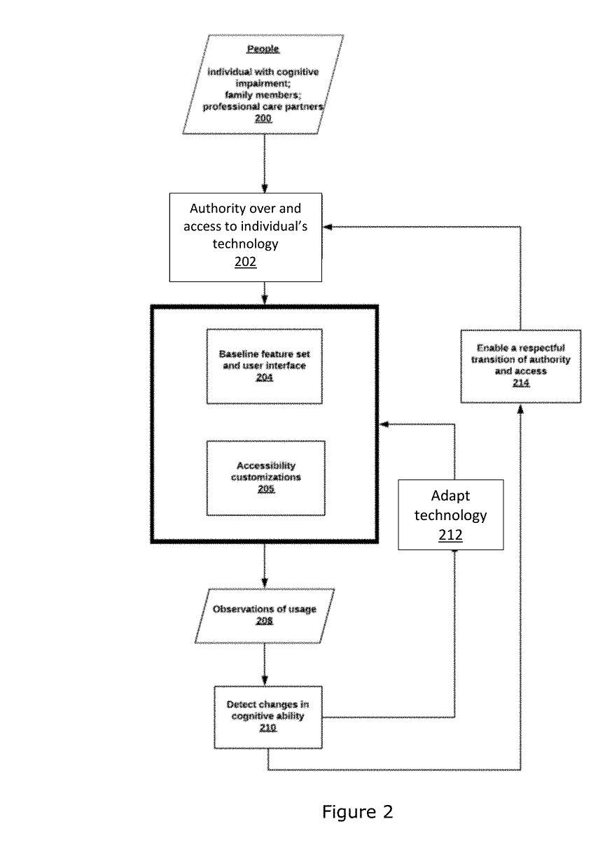

With reference to FIG. 2, example embodiments include systems and methods for:

(1) Identifying people, such as the individual user with cognitive impairment or third-party users such as family members or professional care partners, who may be involved in the plan to designate authority over and access to the individual's technology on user electronic device 100 (as represented in block 200). In example embodiments, this function may be performed by account module 412f as part of an initial user registration process.

(2) Determining and setting authority over and access to the individual's technology (block 202). In example embodiments, an initial authority and access parameters may be set based on user inputs with authorization module 412g, which may then be updated over time based on information or instructions from cognitive adaption system 110. For example, cognitive adaption system 110 may give an individual with mild cognitive impairment exclusive authority over their technology, including deletion of content, font sizes, and access to medication information. In the case of an individual with a severe cognitive impairment, cognitive adaption system 110 may give authority over the individual's technology (in whole or in part) to a third-party user.

(3) Selecting or identifying a baseline set of features and user interface elements according to the individual's present cognitive ability (block 204). In example embodiments, an initial baseline set of features and user interface elements may be set through user setup interactions with UX orchestrator module 412i, which may then update the features and user interface elements automatically over time based on information or instructions from cognitive adaption system 110. For example, cognitive adaption system 110 may give an individual with mild cognitive impairment access to ‘My Health’ features which contain a record of their medical and wellness information and/or their electronic health record such as one accessed through electronic health records system 418 in order that they can use these sources of information when speaking with their doctor.

(4) Choosing suitable accessibility customizations to the individual's technology (block 205). In example embodiments, an initial set of accessibility customizations may determined through user setup interactions with UX orchestrator module 412i, which may then update the accessibility customizations automatically over time based on information or instructions from cognitive adaption system 110. For example, cognitive adaption system 110 may select larger font sizes for an individual with a sight impairment.

(5) Detecting and observing usage of the individual's technology (block 208). A number of examples of observing and detecting a user's interactions with user electronic device 100 are described below. For example, cognitive adaption system 110 may track, via user detection module 112, the number of misspellings in text entered by the individual via user electronic device 100 as the individual engages with the user support system 90 and store this observation in database 114.

(6) Detecting changes in the individual's cognitive ability based on observations (block 210). For example, through analysis module 116, cognitive adaption system 110 may detect a significant increase in the rate of misspellings in text entered by the individual and a significant increase in the rate of slurred speech within speech recorded by the individual by a recording device or microphone based on data in database 114, leading to a conclusion that the individual has progressed to a more severe stage of cognitive impairment. Examples are discussed in detail below.

(7) Adapting the individual's technology in response to a detected change in the individual's cognitive ability (block 212). For example, through analysis module 116, cognitive adaption system 110 may trigger user system adjustment module 118 to cause authorization module 412g to remove access to medical and wellness information and electronic health records through user electronic device 100 when analysis shows that the individual has progressed to a more severe stage of cognitive impairment.

(8) Enabling a respectful transition of authority over and access to the individual's technology in response to a detected change in the individual's cognitive ability (block 214). For example, through analysis module 116, cognitive adaption system 110 may trigger a user system adjustment module 118 and a third-party adjustment module 122 to grant a family member, and/or professional care partner access to medical and wellness information and electronic health records in database 114 when analysis shows that the individual has progressed to a more severe stage of cognitive impairment. Enabling transition of authority and access from the individual user of user electronic device 100 to a third party user of third-party electronic device 102 may for example be done according to legal permissions and rules contained in database 414.

### Detecting Cognitive Change

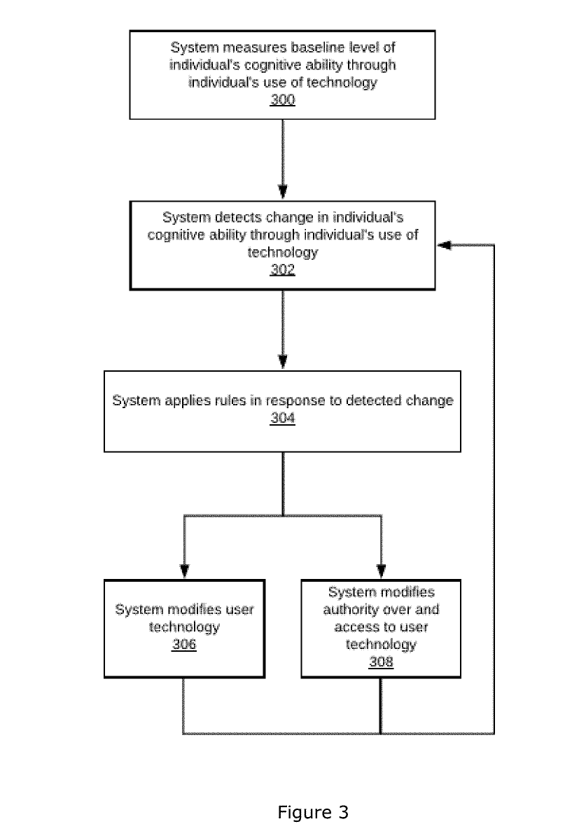

Further detail will now be provided regarding example observations (block 208) and analysis (block 210) which may detect a change in the individual's cognitive ability, with reference to FIG. 3.

A change in the individual's cognitive ability may be detected through a change in one or more of the individual's abilities as follows: (1) an individual's ability to communicate verbally and through written means (for example, word selection, misspellings, and slurring of speech) may change with a change in the individual's cognitive ability; (2) an individual's fine motor control (for example, ability to select a touch target) may change with a change in the individual's cognitive ability; (3) an individual's ability to consume written or auditory information (for example, following simple instructions) may change with a change in the individual's cognitive ability; (4) an individual's ability to complete tasks (for example, recording one's address) may change with a change in the individual's cognitive ability.

With further reference to FIG. 3, in an example embodiment, a change in the individual's cognitive ability may be detected while he/she interacts with technology via, for example, the user electronic device 100. In example embodiments, the individual's interaction with technology is monitored based on the individual's interaction with user interfaces presented on user electronic device 100 during the normal operation of user support system 90. As will be described in greater detail below, user support system 90 includes user support functions that the individual can access and rely on as part of everyday life. By monitoring the individual's interactions with user interface functions that have a purpose other than simply testing the user, detection and analysis of the individual's interaction with technology can be performed in a non-intrusive manner. This may, in at least some cases, mitigate the impact of any observer effect that may arise if the individual is aware that he or she is performing tasks specifically for evaluation purposes.

A baseline can be set, in example step 300, by tracking the individual's interactions of significance with user electronic device 100 and as further disclosed below. A change in the individual's cognitive ability may be detected, in example step 302, based on further observations of the individual's interactions of significance with user electronic device 100. In this regard, the cognitive adaption system 110 receives and stores (for example in database 114) data about a first set of user interactions with the user electronic device 100, and then at a later time receives data about a further set of user interactions with the electronic user device 100. By comparing the data for predetermined types of changes between the first set of user interactions and the further set of user interactions, a change in the user's cognitive ability can be detected. In some examples, the data for the further set of user interactions is re-obtained at on an on-going basis, for example at predetermined time intervals, until an change in cognitive ability is detected. Then, once the individual's technology is adapted to account for the change, new baseline data for the first set of user interactions is obtained.

Example interactions of significance include (but are not limited to) each of the following example interactions:

(1) Selection of input method. In example embodiments, the individual is able to interact with system 90 through user electronic device 100 using text input, for example via a text input device such as a touch screen, keyboard, mouse, touch pad, electronic pen or another type of text input device on a user electronic device 100, or using speech input, for example, via a speech input device such as a microphone or another type of speech input device on a user electronic device 100. Cognitive adaption system 110 may detect, through analysis performed by analysis module 116 based on current user interaction data received through user system detection module 112 and past user data stored in database 114 that an individual has changed their preferred input method or preferred input device as their cognitive ability changes. As examples, cognitive adaption system 110 may detect a change in the individual's preferred input method from text input to speech input, or vice versa, or in their preferred input device from one text input device to another text input device or from one speech input device to another speech input device.

(2) Quantity and quality of input added. As explained in further detail below, in example embodiments, cognitive adaption system 110 may detect, through analysis performed by analysis module 116 based on current user interaction data received through user system detection module 112 and past user data stored in database 114, that the quality and/or quantity of the individual's input to user electronic device 100 has changed as their cognitive ability changes. For example, cognitive adaption system 110 may detect a change in the number of the individual's entries using a touch screen, keyboard, mouse, touch pad, electronic pen, microphone or another type of input device entries.

(3) Successful/failed attempts at location-specific actions. In example embodiments, cognitive adaption system 110 may detect, through analysis performed by analysis module 116 based on current user interaction data received through user system detection module 112 and past user data stored in database 114, that the individual's ability to carry out location-specific actions on user electronic device 100 has changed as their cognitive ability changes. For example, cognitive adaption system 110 may detect a change in the number of the individual's failed attempts to perform a location-specific action such as selecting the ‘back’ button which is displayed at a fixed point on a touch-enabled user interface.

(4) Random and repeated selection of events, bounce rate. In example embodiments, cognitive adaption system 110 may track each event that the individual performs when interfacing with user electronic device 100 within the technology (e.g. while interacting with user interfaces presented by user support system 90), the duration of the event, and the time that the event occurred. Cognitive adaption system 110 may detect via user system detection module 112, database 114, and analysis module 116, that the number of random and/or repeated events carried out by the individual on user electronic device 100 has changed as their cognitive ability changes. For example, cognitive adaption system 110 may detect a change in the number of the individual's successive “enter and back events” (for example, entering a particular screen and then immediately exiting the screen) without performing any other actions between the two events. The rate at which an individual performs successive enter and back events without performing any other actions between them may be referred to as “bounce rate”.

(5) Time taken to perform events and time between events. In example embodiments, cognitive adaption system 110 may maintain a record of the time in minutes and seconds that the individual takes to perform or complete an event after initiating the event. Cognitive adaption system 110 may detect via a user system detection module 112, a database 114, and an analysis module 116, that the amount of time the individual takes to perform an event and/or the time between two events on user electronic device 100 has changed as their cognitive ability changes. For example, a cognitive adaption system 110 may detect a change in the individual's time in seconds to complete an event such as adding ten characters of text input via a keyboard.

(6) Patterns of use. In example embodiments, cognitive adaption system 110 may maintain a record of the events completed by an individual. Cognitive adaption system 110 may detect via user system detection module 112, database 114, and analysis module 116, that the individual's pattern of use of user electronic device 100 has changed as their cognitive ability changes. For example, cognitive adaption system 110 may detect a change in the number of entries such as daily events added by the individual. Further, for example, cognitive adaption system 110 may detect a change in the number of interactions to view information such as medical and wellness information and electronic health records available to the individual through the individual's user electronic device 100.

(7) Use of support. In example embodiments, cognitive adaption system 110 may provide technical support to the individual in the form of audio support or through text. Cognitive adaption system 110 may detect via user system detection module 112, database 114, and analysis module 116, that the frequency that an individual accesses support content or the individual's preference for audio support over text support through user electronic device 100 has changed as their cognitive ability changes. For example, cognitive adaption system 110 may detect a change in the number of the individual's attempts to access support or the time in seconds that the individual spends accessing support.

### Quality of Input

Specific examples of the operation of cognitive adaption system 110 to detect cognitive changes based on changes in quality of input will now be described. In example embodiments, the individual is able to add free form content, for example via text input devices such as a touch screen, keyboard, mouse, touch pad, electronic pen or any other type of text input device, or add speech content, for example, via a speech input device such as a microphone via user electronic device 100. In example embodiments, cognitive adaption system 110 is configured to detect detect via user system detection module 112, database 114, and analysis module 116 that the quality of free form content in a second plurality of inputs added by the individual has changed from the quality of free form content in a first plurality of inputs added by the individual. The change is detected during a period of time when the individual's cognitive ability may be changing. Significant features of the content added by the individual that may be tracked and analyzed via cognitive adaption system 110 include (but are not limited to):

(1) Vocabulary size. In example embodiments, cognitive adaption system 110 may detect via user system detection module 112, database 114, and analysis module 116 that the individual uses a smaller or larger variety of words or fewer or more complex words in a second plurality of free form content added through user electronic device 100 compared to a first plurality of free form content added through user electronic device 100. In example embodiments, complex words may be defined as words having three or more syllables. A change may be detected via text content added through a text input device or speech content added via a recording device or microphone by the individual as their cognitive ability changes.

(2) Word specificity. In example embodiments, cognitive adaption system 110 may detect via user system detection module 112, database 114, and analysis module 116 that the individual uses an increasing or decreasing frequency of vague or indefinite words in a second plurality of free form content added through user electronic device 100 compared to a first plurality of free form content added through user electronic device 100. Vague or indefinite words may include “thing”, “stuff”, “good”, “bad”, and “nice”. A change may be detected via text content added through a text input device or speech content added via a recording device or microphone by the individual as their cognitive ability changes.

(3) Use of fillers and mispronunciations. In example embodiments, cognitive adaption system 110 may detect via user system detection module 112, database 114, and analysis module 116 that the individual uses an increasing or decreasing frequency of filler words, slurred speech, or word mispronunciations in a second plurality of free form content added through user electronic device 100 compared to a first plurality of free form content added through user electronic device 100. Filler words may include “um” and “er”. A change may be detected via text content added through a text input device or speech content added via a recording device or microphone by the individual as their cognitive ability changes.

(4) Occurrence of repeated words and phrases. In example embodiments, cognitive adaption system 110 may detect via user system detection module 112, database 114, and analysis module 116 that the individual uses an increasing or decreasing frequency of repeated words or groups of words in a second plurality of free form content added through user electronic device 100 compared to a first plurality of free form content added through user electronic device 100. A change may be detected via text content added through a text input device or speech content added via a recording device or microphone by the individual as their cognitive ability changes.

(5) Proportion of word classes. In example embodiments, cognitive adaption system 110 may detect via user system detection module 112, database 114, and analysis module 116 that the individual uses a decreasing or increasing relative frequency of some word classes in a second plurality of free form content added through user electronic device 100 compared to a first plurality of free form content added through user electronic device 100. Word classes may, for example, be defined as adjectives, adverbs, and pronouns. A change may be detected via text content added through a text input device or speech content added via a recording device or microphone by the individual as their cognitive ability changes.

(6) Spelling accuracy. In example embodiments, cognitive adaption system 110 may detect via user system detection module 112, database 114, and analysis module 116 that the individual uses an increasing or decreasing frequency of misspelled words in a second plurality of free form content added through user electronic device 100 compared to a first plurality of free form content added through user electronic device 100. A change may be detected via text content added by the individual through a text input device when there is no automatic spelling assistance as their cognitive ability changes.

(7) Syntactic complexity. In example embodiments, cognitive adaption system 110 may detect via user system detection module 112, database 114, and analysis module 116 that the individual uses a decreasing or increasing frequency of punctuation, spaces, other writing conventions, and/or complex sentences in a second plurality of free form content added through user electronic device 100 compared to a first plurality of free form content added through user electronic device 100. Punctuation may include, for example, proper use of commas, apostrophes, and questions marks. Other writing conventions may include, for example, proper use of capital letters and grammar. A complex sentence may be a sentence that includes an independent clause and at least one dependent clause. A change may be detected via text content added through a text input device by the individual as their cognitive ability changes.

(8) Text sentiment analysis. In example embodiments, cognitive adaption system 110 may detect via user system detection module 112, database 114, and analysis module 116 that the individual has experienced a change in mood, mindset, or outlook in a second plurality of free form content added through user electronic device 100 compared to a first plurality of free form content added through user electronic device 100. A change may be detected via semantic analysis of text content added through a text input device or speech content added via a recording device or microphone by the individual as their cognitive ability changes.

(9) Appropriate diction. In example embodiments, cognitive adaption system 110 may detect via user system detection module 112, database 114, and analysis module 116 that the individual uses an increasing or decreasing frequency of contextually incorrect words, a different tone of the language, and/or a different choice of input language in a second plurality of free form content added through user electronic device 100 compared to a first plurality of free form content added through user electronic device 100. Contextually incorrect words may include the word “book” where the word “newspaper” is contextually correct. Tone of the language may be formal or colloquial. Choice of input language may be English or French. A change may be detected via text content added through a text input device or speech content added via a recording device or microphone by the individual as their cognitive ability changes.

(10) Correctness of word order. In example embodiments, cognitive adaption system 110 may detect via user system detection module 112, database 114, and analysis module 116 that the individual uses an increasing or decreasing frequency of misplaced words within a sentence and/or awkward word structure in a second plurality of free form content added through user electronic device 100 compared to a first plurality of free form content added through user electronic device 100. Misplaced words within a sentence may include the phrase “I book a read” instead of “I read a book”. Awkward word structure may include “today I went to church and am eating dinner”. A change may be detected via text content added through a text input device or speech content added via a recording device or microphone by the individual as their cognitive ability changes.

(11) Appropriate response for solicited input. In example embodiments, cognitive adaption system 110 may detect via user system detection module 112, database 114, and analysis module 116 that the individual is increasingly more or less able to respond appropriately to solicited input in a second plurality of free form content added through user electronic device 100 compared to a first plurality of free form content added through user electronic device 100. For example, the individual may provide an appropriate or inappropriate response when asked to enter their contact information or name of their spouse. A change may be detected via text content added through a text input device or speech content added via a recording device or microphone by the individual as their cognitive ability changes.

In example embodiments, cognitive adaption system 110 may track the interactions of significance listed above to assess the baseline of the individual's cognitive ability in example step 300. In example step 302, a system may detect a change via user system detection module 112, database 114, and analysis module 116 through a second plurality of inputs added through user electronic device 100 compared to the baseline in example step 300 or to a different plurality of inputs added through user electronic device 100 after the baseline and before the second plurality of inputs.

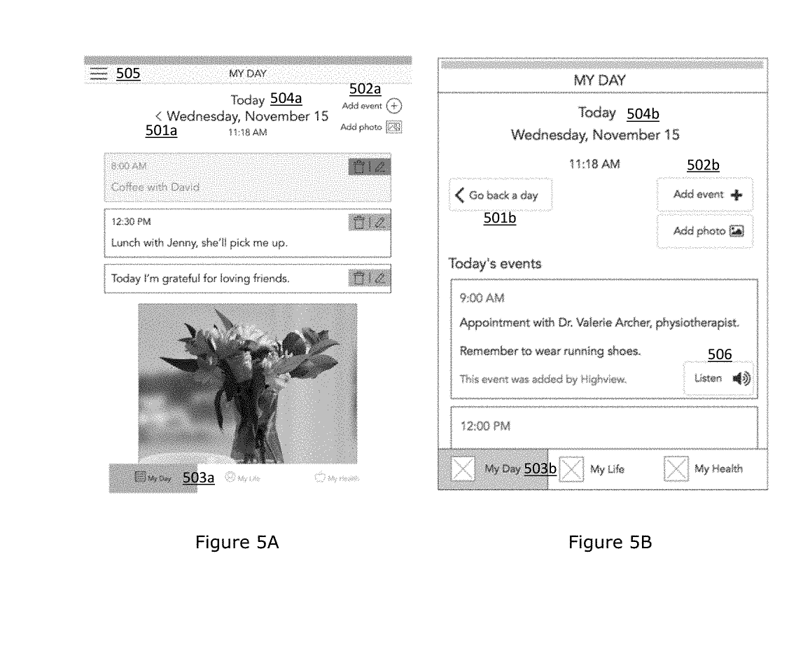

In example embodiments, assessments in example steps 300 and 302 are non-obtrusive in that they are performed by cognitive adaption system 110 while the individual uses a technology for its intended function. By way of example, the user interactions that are used to detect changes are the user interactions that occur in respect of user interfaces presented on user electronic device 100 during operation of user support system 90. Examples of these user interfaces are discussed in greater detail below in respect of FIGS. 5A to 11. Accordingly, in example embodiments, cognitive adaption system 110 does not prompt the individual to complete any additional tasks or provide any additional information. Further, assessments are non-disruptive in that they do not appear to the individual or affect the user interface or features in any way. These aspects are important to ensure that assessments do not cause any disruption or anxiety to the individual's experience with a technology.

In example embodiments, assessments in example steps 300 and 302 are performed by cognitive adaption system 110 by analysis module 116 based on successive pluralities of inputs added by the individual and stored in a database 114. For example, an analysis module 116 may analyze successive instances of the interactions of significance and/or content features added by the individual, for example vocabulary size and word specificity, through multivariate statistical methods, for example multivariate change point analysis. Analysis module 116 may analyze successive instances of the interactions of significance and/or content features added by the individual through machine learning methods. An output of analysis module 116 is an appropriate rule in response to a detected change. Cognitive adaption system 110 applies rules in response to a detected change in example step 304 through user adjustment module 118 and third-party adjustment module 122.

In example embodiments, some interactions of significance and content features listed above may be more informative than others. A rule in example step 304 may be based on a detected change in a single interaction of significance or content feature. A rule in example step 304 may be based on a detected change in more than one interaction of significance and/or content feature. The criteria placed on various interactions of significance or content features within a rule are given in analysis module 116 and may be changed by a system administrator at any time. In example embodiments, analysis methodology and criteria that define rules within analysis module 116 are based on leading scientific research on the effect of an individual's cognitive decline on their use of technology measured through the interactions of significance and content features described above. For example, scientific research shows that an individual's cognitive decline results in a sharp decrease in the size of vocabulary that is used by the individual. In an example embodiment, cognitive adaption system 110 detects a change in the individual's vocabulary size through features of the content added by this individual via a user electronic device 100.

In example embodiments, a rule in example step 304 may direct cognitive adaption system 110 to generate a report or a recommendation for output by the user electronic device 100 and/or a third-party electronic device 102, for example one that belongs to a care manager, family member, and/or professional care partner. For example, an analysis module 116 may detect that the individual is no longer accessing a particular feature, for example electronic health records available through user electronic device 100. A rule in example step 304 may direct cognitive adaption system 110 to generate a report for the third-party electronic device 102 noting this change and a recommendation to the individual and/or third party that this particular feature (or access to this feature) should be removed from user electronic device 100. A report may include various indicators and graphs of data over time. Rules for creating reports and recommendations may be tailored to the preferences of the individual and/or one or more third-party members.

In example embodiments, reports and recommendations discussed above may be communicated through a system administration module 120.

The example embodiments described above contemplate that changes in cognitive ability are detected based on detected changes between sets of user interactions that occur at different times. However, in some example embodiments a detected change in cognitive ability may be based only on the user's current interactions with the user electronic device 10. For example, if a user is unable to meet certain thresholds when interacting with a current user interface, an implicit assumption can be made that cognitive ability has changed to the point that a technology adaptation is required. Accordingly, in some examples, a decision that the individual's technology needs to be adapted according to a current state of the individual's user cognitive ability may be determined based only on a current set of user interactions. In such a system, cognitive ability would be determined based on a function/algorithm applied to the current set of user interactions only. Based on the output of the function a decision can be made if the individual's technology needs to be adapted for the current cognitive ability level of the user. The function/algorithm could be rules based, or it could be learned. By way of example, a rules-based function could be configured to determine that the occurrence of a predetermined number and/or pattern of failed location-specific actions indicates that the individual's current cognitive ability is at a level that does not correspond to the individual's technology, thereby indicating that a technology adaptation is required.

### Adapting Technology to Changes in Cognitive Ability

With reference to FIGS. 5 through 10, further example detail will now be provided regarding adapting the individual's technology, such as the user experience and features available through user electronic device 100, when a change in the individual's cognitive ability is detected as given above.

It may be beneficial for an individual experiencing cognitive impairment to use technology with features that support their needs and a user interface that is manageable given the individual's ability at a given point in time. The needs and ability of an individual may change as time progresses and cognitive ability declines (for example, if the individual suffers from dementia) or as cognitive ability improves (for example, if the individual is recovering from a TBI (tramautic brain injury)). An individual with cognitive decline may stop using technology when the technology feature set is no longer relevant or the user interface is too complicated given their ability at a given point in time. Alternatively, an individual with cognitive improvement may increase their use of technology when the technology feature set and user interface is relevant to their ability at a given point in time.

Accordingly, in example embodiments, as disclosed in example FIGS. 5 to 10, in example embodiments cognitive adaption system 110 is configured to automatically modify elements of a feature set and user interface according to rules upon detecting threshold levels of a change in cognitive ability as in example step 306.

Example embodiments refer to elements of a technology at one of three levels. As cognitive adaption system 110 detects a change in the individual's cognitive ability through their use of a user electronic device 100, it may adapt one or more technology elements available through user electronic device 100 from one level to a different level. The levels are referred to as levels 1, 2, and 3, relating, in order, to adaptations that are made for an individual with decreasing cognitive ability. Example feature sets, technical support, and user interface elements for user support system 90 are described below for each of levels 1, 2, and 3, which, at a general level, are appropriate for an individual who has either mild, moderate, or severe cognitive impairment, respectively. User device adaptations need not be made as a group and may be made singly or in smaller groups according to prescribed rules in cognitive adaption system 110 applied to successive pluralities of inputs as discussed above. For example, analysis module 116 may detect that in a second plurality of inputs, the individual is no longer accessing a particular feature, for example medication information, available through user electronic device 100 compared to a first plurality of inputs. A rule in example step 304 may direct cognitive adaption system 110 to adapt this feature from level 1 to level 2, which would involve a removal of this feature from (or access to this feature through) user electronic device 100. Other contemporaneous changes may or may not be made.

In further alternative embodiments, a different number of levels may be used, for example, more than three levels or fewer than three levels.

Example embodiments of elements of a level 1 technology in the context of user interfaces presented by user support system 90 are disclosed in FIGS. 5A, 6A, 7A, 8A, 9A, and 11. For an individual with mild cognitive impairment, a primary goal of a technology solution may be to promote independence and secondary goals of a technology solution may be to promote productivity and the ability to create, consume, share, and store information, and to facilitate communication with professional care partners, friends, and family. Elements of a level 1 technology may be suitable for an individual with mild cognitive impairment.

Example embodiments of elements of a level 2 technology in the context of user interfaces presented by user support system 90 are disclosed in FIGS. 5B, 6B, 7B, 8B, and 9B. For an individual with moderate cognitive impairment, a primary goal of a technology solution may be to promote assisted independence and secondary goals of a technology solution may be to increase safety, to promote the ability to consume information, to facilitate communication with professional care partners, friends, and family, and to provide comfort to the individual. Elements of a level 2 technology may be suitable for an individual with moderate cognitive impairment.

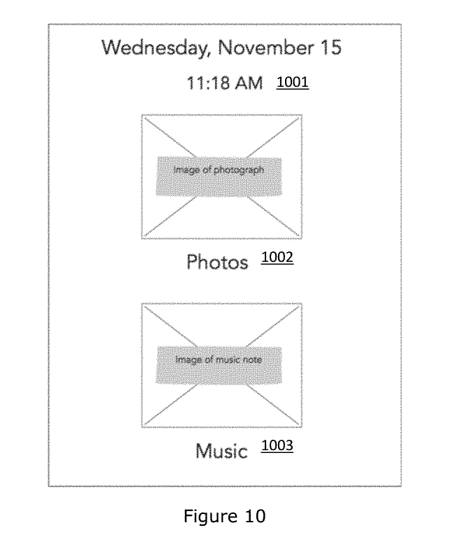

An example embodiment of elements of a level 3 technology in the context of a user interface presented by user support system 90 is disclosed in FIG. 10. For an individual with severe cognitive impairment, a primary goal of a technology solution may be to provide comfort to the individual and secondary goals of a technology solution may be to promote an ability to consume information. Elements of a level 3 technology may be suitable for an individual with severe cognitive impairment.

As cognitive adaption system 110 detects a change in an individual's cognitive ability, which may be either a decline or improvement in cognitive ability, in example embodiments, cognitive adaption system 110 automatically modifies elements of the technology, including (1) the technology feature set, (2) technical support for the user, and (3) user interface design, as follows. References are made to elements of FIGS. 5 through 10 for an example embodiment.

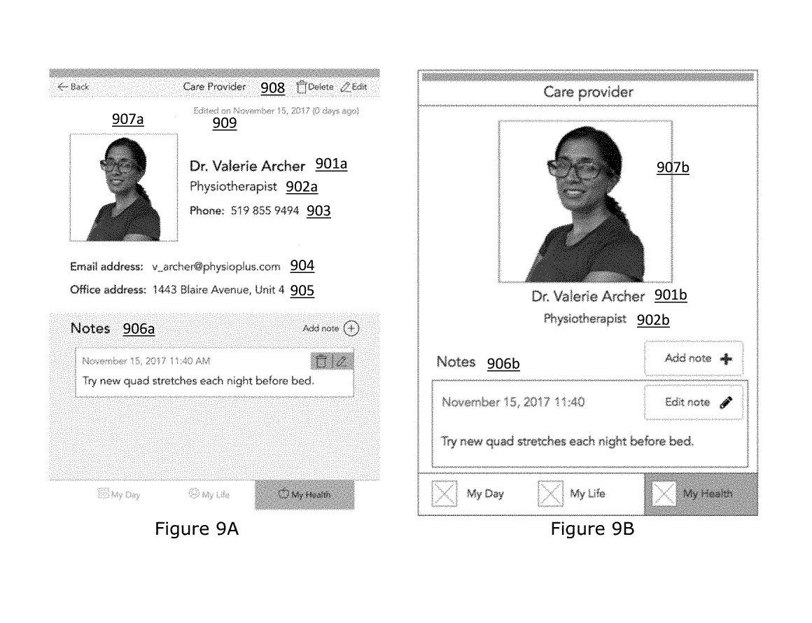

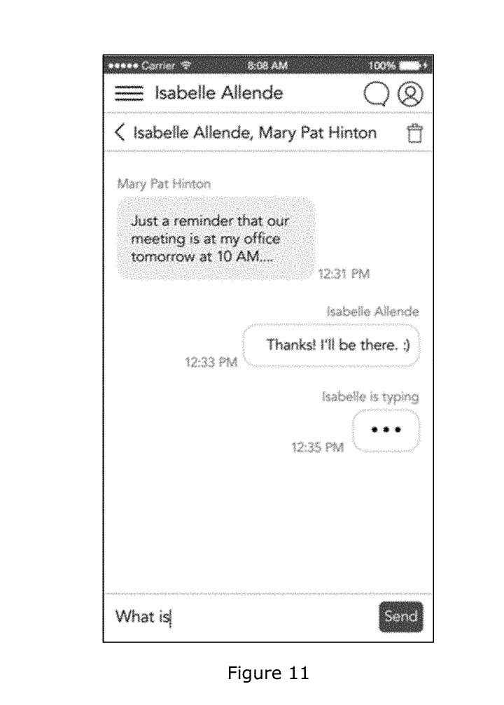

(1) Technology feature set. In example embodiments, level 1 technology feature set elements may be suitable for an individual with mild cognitive impairment. At level 1, features are available for the individual to access, add, edit, and delete:
‘My Day’ content, particularly events in a daily agenda (FIG. 5A) (a user interface for a calendar/event scheduling feature of user support system 90 is shown);
‘My Life’ content, particularly photo albums (601a, 602a, 603, 604, 605, 606), personal photos (701a, 702a, 703, 704), photo captions, audio recordings for photos, personal notes, how-to information, lists of preferred activities, and personal notifications; (photo viewing user interfaces for a media viewing feature of user support system 90 are shown)
‘My Health’ content, particularly medications and supplements (801), personal health notes (802), family health history (803) care team details (804a, FIG. 9A), and a care journal (805a); (personal information viewing and editing interfaces of user support system 90 are shown)
‘Chat’ content, particularly messages within a conversation (FIG. 11) (an electronic messaging user interface for a messaging feature of user support system 90 is shown)

In example embodiments, level 2 technology feature set elements may be suitable for an individual with moderate cognitive impairment. At level 2, features are available for the individual to access:
‘My Health’ content, particularly care team details (804b, FIG. 9B) and a care journal (805b),
‘My Day’ content, particularly personal events in daily agenda (FIG. 5B) and events pushed to daily agenda by a care facility (506),
‘My Life’ content, particularly photo albums (601b, 602b), personal photos (701b, 702b), photo captions, audio recordings for photos, personal notes, how-to information, preferred activities, activity details pushed by a care facility, interest packages (for example, curated photos, music, videos), personal notifications, and notifications pushed by a care facility.

The individual with cognitive impairment may be able to add, edit, or delete content depending on a transfer of authority plan. Some content and features may be hidden if cognitive adaption system 110 detects that they are no longer useful for the individual. In example embodiments, level 3 technology feature set elements may be suitable for an individual with severe cognitive impairment. At level 3, features are available for the individual to view or listen to content such as photos (1002), recordings, music (1003), and interest packages.

(2) Technical support for the user. In an example embodiment, technical support content may be available to the individual through a user electronic device 100. At level 1, technical support content is available through a menu (505) and contains text that is available on demand to the individual. At level 2, a user electronic device 100 may serve technical support content to the individual when cognitive adaption system 110 detects that the individual requires assistance. Support may be provided through text and/or audio means and instructions are provided via simple language. At level 3, a user electronic device 100 may serve technical support content to the individual when cognitive adaption system 110 detects that the individual requires assistance. Support may be provided through audio means only and instructions are provided via very simple language.

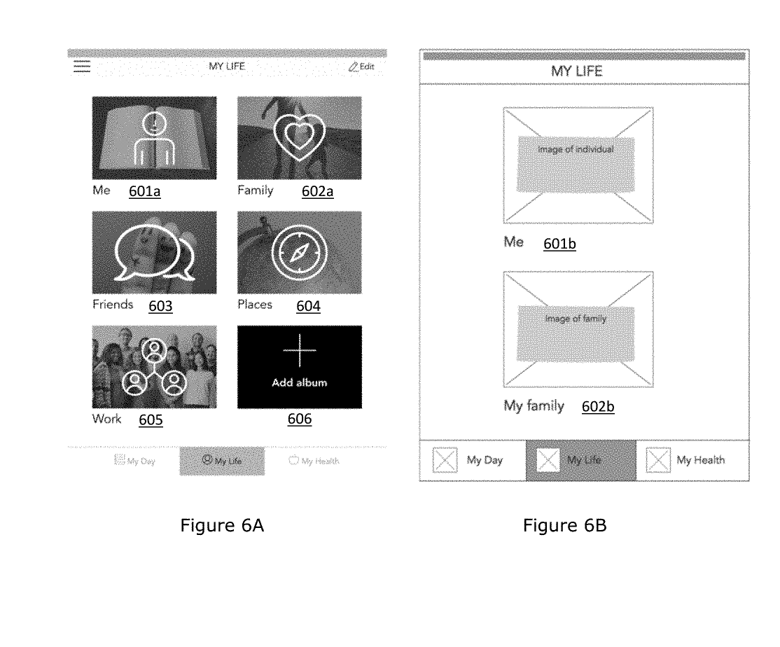

(3) User interface design. In example embodiments, a level 1 user interface design may be suitable for an individual with mild cognitive impairment. At level 1, user interface design reflects the Web Content Accessibility Guidelines (WCAG) level AA guidelines for:
colour contrast,
text labels to accompany images and icons (502a),
button size (large) (501a),
touch target size (503a),
font sizes (minimum 12 pt) (504a),
plain language for text instructions and errors (805a),
text and audio input,
a navigation system based on the native operating system (hence users apply what they already know to a new context),
visual feedback (action success/failure),
number of elements on a page (max 6-8) (FIG. 6A), and
consistent design and interaction patterns throughout the technology.

In example embodiments, a level 2 user interface design may be suitable for an individual with moderate cognitive impairment. At level 2, user experience design reflects WCAG level AAA guidelines for
colour contrast,
text labels to accompany images and icons (501b),
no icons over images (601b),
no abstract icons (601b),
buttons (large) (501b),
touch target size (larger than level 1) (503b),
font size (larger than level 1) (503b),
very simple language for instructions and errors, instructions and help available in text and audio,
text and audio input,
a simplified navigation system,
visual and auditory feedback (706),
minimal elements on a page (max 4-6) (FIG. 6B), and
consistent design and interaction patterns throughout the technology.

In example embodiments, a level 3 user interface design may be suitable for an individual with severe cognitive impairment. At level 3, user experience design reflects WCAG level AAA guidelines for
colour contrast,
simple images to support navigation (1002),
button size (extra-large) (1002),
touch target size (extra-large) (1002),
font size (extra-large) (1001),
minimal (if any) text,
audio input,
very simple navigation system,
auditory feedback,
very few elements on a page (max 1-2) (FIG. 10), and
an option to view content on loop.

In example embodiments, when cognitive adaption system 110 detects a decline in an individual's cognitive ability, elements of a technology may adapt from level 1 to level 2 as follows: images are enlarged; font sizes are enlarged; content is simplified; white space is added around interface elements and text; visual affordance for interaction elements such as buttons is increased; fewer elements appear on a screen at one time; icons overlaying images are removed; images are made more literal and less abstract; navigation menus to access peripheral content such as account information are removed; delete functions are removed; navigation bars are enlarged; audio options appear so the individual can listen to content; features are removed. When cognitive adaption system 110 detects an improvement in an individual's cognitive ability, elements of a technology may adapt from a level 2 to a level 1, a reversal of the adaptations described above.

In example embodiments when cognitive adaption system 110 detects a decline in an individual's cognitive ability, a feature set on, or available through, user electronic device 100 may adapt from level 1 to level 2 as follows:
remove features: ‘Friends photo album’ (603), ‘Places photo album’ (604), ‘Work photo album’ (605), ‘Add album’ feature (606), ‘Medications and supplements’ (801), ‘Personal health history’ (802), ‘Family health history’ (803), ‘Care provider's phone number’ (903), ‘Care provider's email address’ (904), and ‘Care provider's office address’ (905), ‘Chat’ functionality (FIG. 11)
retain features: ‘Me photo album’ (601), ‘Family photo album’ (602), ‘My care team’ (804), ‘My care journal’ (805), ‘Care provider's name’ (901), ‘Care provider's title’ (902), and ‘Notes’ (906)
add a feature such as ‘Listen’ (706)

In example embodiments, when cognitive adaption system 110 detects a decline in an individual's cognitive ability, user interface design on a user electronic device 100 may adapt from level 1 to level 2 as follows:
image 907b is larger than image 907a,
date and time of last edit 909 is removed in FIG. 9B,
font size 503b is larger than 503a, there is more white space around 504b than 504a,
there is more separation and outlining around buttons (502b vs. 502a),
a button has more separation, outlining, and text (501b vs. 501a),
there are fewer elements on ‘My Life’ screen (FIG. 6B vs. FIG. 6A),
icons over the images in 601a and 602a are removed in 601b and 602b,
images in 601b and 602b are more literal than images in 601a and 602a,
the navigation menu to access peripheral content such as account information 505 is removed in FIG. 5B,
an option to delete a care provider record 908 is removed in FIG. 9B,
the navigation bar 503b is larger than 503a,
an option to listen to ‘Today's events’ (506) is added.

In example embodiments, when cognitive adaption system 110 detects a decline in an individual's cognitive ability, elements of a technology may adapt from level 2 to level 3 as follows: images are enlarged; font size for text elements are enlarged; a maximum of two elements appear at one time; abstract images are changed to literal images or removed. Technology elements are selected based on scientific research that is directed towards providing comfort to the individual. When cognitive adaption system 110 detects an improvement in an individual's cognitive ability, elements of a technology may adapt from a level 3 to a level 2, a reversal of the adaptations described above.

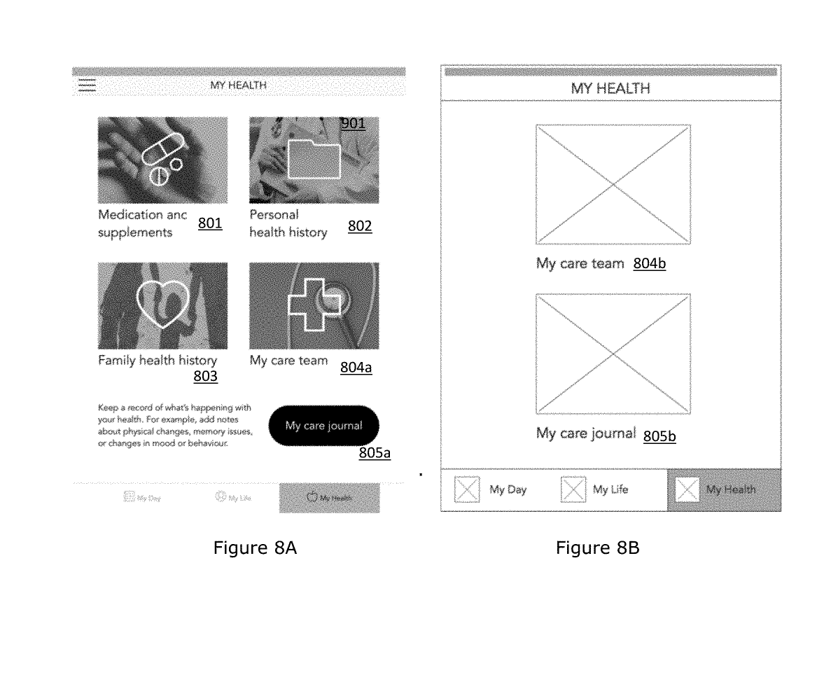

In example embodiments, when cognitive adaption system 110 detects a decline in an individual's cognitive ability, a feature set available on or through user electronic device 100 may adapt from level 2 to level 3 as follows:
remove features: ‘My Day’ (FIG. 5B), ‘My Health’ (FIG. 8B), ‘Notes’ (906b) are removed in FIG. 10
replace features: ‘Me photo album’ (601a) and ‘My family photo album’ (601b) are replaced with ‘Photos’ (1002)
add a feature such as ‘Music’ (1003)

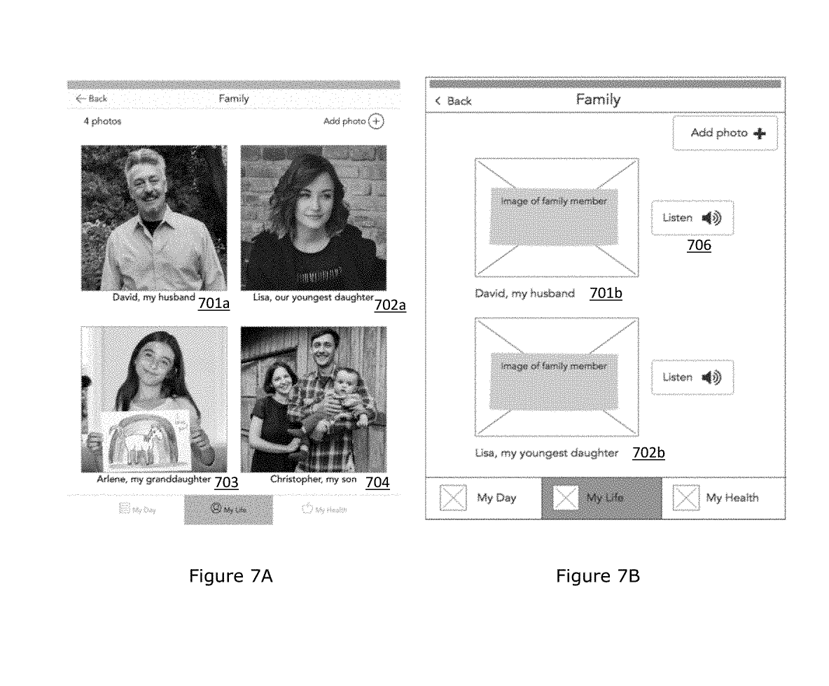

In example embodiments when cognitive adaption system 110 detects a decline in an individual's cognitive ability, user interface design on user electronic device 100 may adapt from level 2 to level 3 as follows:
date and time font size is larger (1001 vs. 504b)
fewer elements appear on a screen (FIG. 10 vs. FIG. 5B, FIG. 6B, FIG. 7B, FIG. 8B, and FIG. 9B)
the navigation bar is removed (FIG. 10 vs. 503b)

In example embodiments, cognitive adaption system 110 may detect that the individual has additional conditions or impairments that make it more difficult for the individual to access features of the technology. For example, a sight impairment may make it more difficult for an individual to interact with a screen on user electronic device 100.

Accordingly, an example embodiment as outlined in FIG. 2 offers accessibility customizations, as example step 206, which are options to customize the technology in order to meet the individual's unique needs. Accessibility customizations may be chosen by the individual, a family member, or a care partner during setup of the technology or at a later time. Further, certain rules within example step 304 based on observations of the individual's interactions with user electronic device 100 may prompt cognitive adaption system 110 to notify the individual, family member, and/or care partner when rules suggest particular accessibility customizations. For example, if cognitive adaption system 110 detects that the individual has a threshold number of missed attempts at location-specific events, for example, missed attempts at pressing a back button on a touchscreen of a user electronic device 100, then cognitive adaption system 110 may notify the individual with a suggestion to increase button sizes of user interfaces presented on user electronic device 100. In an example embodiment, possible accessibility customizations may involve the following features of a user interface: WCAG level AA or level AAA color contrast, button and touch target size, font size, complexity of language, visual and/or audio feedback, number of elements on page, amount of white space around elements, text instructions and/or audio instructions, how and when support content is available, and simplicity of images and icons.

In example embodiments, as noted above, cognitive adaption system 110 is configured to detect one or more changes in an individual's cognitive ability, as disclosed in example step 302, while the individual engages with the technology, and then to adapt the technology to the change(s), as disclosed in example step 306. Cognitive adaption system 110 may be configured to detect a change based on a difference between multiple pluralities of inputs that is greater than some defined threshold value. Alternatively, cognitive adaption system 110 may be configured to detect a change based on analytical methods, such as machine learning techniques or multivariate change point analysis techniques, that are applied to multiple pluralities of inputs. For example, cognitive adaption system 110 may calculate a score based on interactions such as vocabulary size, use of fillers and mispronunciations, occurrence of repeated words and phrases, proportion of word classes, and spelling accuracy via text content added by the individual via user electronic device 100 within the latest week. A rule of example step 304 of FIG. 3 could be applied according to whether a score is statistically significantly different than a similarly calculated score in previous weeks. In another example, cognitive adaption system 110 may detect a change in the frequency that an individual adds text content into a particular feature of the technology, such as the ‘My Day’ feature of the user support system 90 (FIG. 5A). A rule could depend on whether the time between a particular event in the present week is statistically significantly different than the time between that particular event in previous weeks.

Changes to the technology are implemented in a way that minimizes disruption and confusion to the user of the technology according to their present cognitive ability. In an example embodiment, the technology may increase font sizes and sizes of images in response to an increase in a rate of failed attempts at location-specific events, such as selecting a back button on a user electronic device 100. A rule within example step 304 in FIG. 3 may instruct cognitive adaption system 110 to recommend a change to the individual, family member, and/or care partner and request their authorization to make the change. Following authorization, a rule may instruct cognitive adaption system 110 to send a notice of when the change will occur and what impact it will have on the feature set and user interface of user electronic device 100. Following implementation of the change, a rule may instruct cognitive adaption system 110 to offer change support to the individual via a user electronic device 100 or to a family member and/or care partner via a third-party electronic device 102. Rules are set purposefully and precisely to minimize user confusion or disruption with consideration for the present state of the individual's cognitive ability as detected by cognitive adaption system 110.

### Respectful Transition of Authority Over Technology and Data

Example embodiments of cognitive adaption system 110 are configured to adaptively and automatically control third-party authority and access via, for example, third-party electronic device 102, to an individual's technology, for example access to features of user support system 90 through user electronic device 100, based on a detected change in cognitive ability. Cognitive adaption system 110 is configured based on the recognition that the suitability and need for another person to have authority and access over an individual's technology may change depending on the cognitive ability of an individual. Considerations of control, data privacy, and information security are relevant. Further, consideration of respect for the individual who may be relinquishing authority and access is relevant.

As noted above, the ability for an individual to manage their daily life independently may change as their cognitive ability declines, for example, if the individual suffers from dementia, or as their cognitive ability improves, for example, if the individual is recovering from a TBI. An individual with cognitive decline will tend to require more assistance in daily tasks, for example, in managing their medications, and will tend to require more third-party authority over and access to their technology, for example, their medical history, as time passes. Some individuals with cognitive decline will tend to find their information, for example, their care plan, upsetting and will tend to have more peace of mind when a care manager or care partner manages this information on their behalf. An individual with cognitive improvement will tend to require less assistance in daily tasks and will tend to require less third-party authority over and access to their technology as time passes. Adaptive third-party authority over and access to their technology that is appropriate for their present cognitive ability can provide advantages of safety, independence, and peace of mind for an individual.

Accordingly, in example embodiments, upon detecting a change in an individual's cognitive ability, authority over and access to an individual's technology is transitioned to or from the individual and one or more of their care manager, family members and/or care partners with respect in example step 308.

In example embodiments, when an individual is experiencing cognitive decline or improvement, transition of authority over an individual's technology is managed with respect by following a process including the following components:

(1) Plan A plan or report may include a list of the elements of the technology that will transition, trigger points for the transitions, and a transition process. In an example embodiment, a plan may state that access to the individual's medication information is to be given to a family member at the timepoint when cognitive adaption system 110 detects that the individual has not accessed the medication information for a period of 60 days. The transition process may state that no authorizations are required for this aspect of the plan. The individual is given the opportunity to create a plan in conjunction with a family member or caregiver at an early stage of their cognitive decline. Cognitive adaption system 110 may offer a guide to help create a plan. For example, cognitive adaption system 110 may send via user electronic device 100 and third-party electronic device 102, an outline of why a plan is important, a step-by-step set of questions to answer, options for trigger conditions for the transitions, options for who should authorize the transitions, and a recommendation for these items based on the cognitive ability of the individual.

(2) Invitations Cognitive adaption system 110 may detect when trigger conditions that are defined within a plan are met and send invitations to the individual via a user electronic device 100 and a care manager, family member and/or care partner involved via third-party electronic device 102. In an example embodiment, when cognitive adaption system 110 detects that the individual has not accessed his/her medication information for a period of 60 days, then cognitive adaption system 110 may send an invitation to a family member for access to the individual's medication information. In a further example embodiment, when cognitive adaption system 110 detects that the individual who has not accessed his/her medication information previously then accesses his/her medication history every day for a week, then cognitive adaption system 110 may send an invitation to the individual and the family member to revoke the family member's access to the individual's medication information.

(3) Sign-offs Cognitive adaption system 110 modifies authority over and access to the individual's technology once the appropriate authorization that are defined within the plan are received via either a user electronic device 100 or third-party electronic device 102. For example, a plan may specify that authorization is required solely from the individual in order for a family member to be given access to the individual's medication history. In a further example, a plan may specify that authorization is required from both the individual and a family member in order for a family member's access to the individual's medication history to be revoked.

In example embodiments, a plan may designate that multiple authorizations are required in order to execute particular events within the technology. For example, in the case where the individual has a severe cognitive impairment, a plan may specify that both the individual and a family member may be required to authorize a particular event within the technology such as deletion of content.

In example embodiments, a change in an individual's cognitive ability may be detected while he/she interacts with a technology for its usual function. The trigger points within a plan may be based on a detection of a change in the individual's cognitive ability via cognitive adaption system 110.

The process in FIG. 3, whereby cognitive adaption system 110 detects a change in an individual's cognitive ability through the individual's interactions with the technology (302), applies rules in response to a detected change (304), and modifies the technology (306) and authority over the technology (308), is continuous. The process operates as long as the individual uses the technology.

In example embodiments, a solution that detects a change in an individual's cognitive ability, adapts a technology in response to a detected change, and modifies a technology and authority over a technology may mitigate one or more of the following problems: an individual experiencing cognitive impairment needs complete and accurate health care information; an individual experiencing cognitive impairment needs memory aids for added independence and dignity; an individual experiencing cognitive impairment gains comfort from recollections of past activities or important people; an individual experiencing cognitive impairment wants to communicate electronically with a family member who lives remotely; a family member wants to consume content relating to the individual's activities, care plan, and health and wellness when the family member lives remotely; a professional care partner wants to communicate with the family and other care partners about the individual's care and consume content relating to the individual's personal preferences and personal history in order to improve the quality of person-centered care they provide; an individual needs to give access and authority for their information to their Power of Attorney at a suitable point during their cognitive decline; paper records and memory aids are time-consuming to create, hard to update, easy to lose, and may degrade over time; and an individual with a cognitive impairment finds other technology solutions difficult to use and/or insufficient for their needs.

In example embodiments, a solution to the previous needs may function as one or more the following: a digital record of health care and other vital information for an individual with cognitive impairment; a digital memory aid with content added solely by the individual or jointly by the individual and a family member, care manager, and/or professional care partner; a record of the individual's personal history and personal preferences that gives a care manager or professional care partner access to information that can be used to deliver person-centered care; a record of the individual's activities, care plan, and health and wellness information that can be accessed by a family member who lives remotely; a chat tool that is suitable for an individual who wants to communicate with a family member who lives remotely; a chat tool that is suitable for a care partner who wants to communicate with an individual's family or other care partners; a comfort item for an individual with advanced cognitive impairment; a tool to share content to or from an individual with a cognitive impairment and a family member, care manager, and/or professional care partner; a tool to manage the transition of access and authority over an individual's information at an appropriate point of an individual's cognitive decline.

The embodiments of the present disclosure described above are intended to be examples only. The present disclosure may be embodied in other specific forms. Alterations, modifications and variations to the disclosure may be made without departing from the intended scope of the present disclosure. While the systems, devices and processes disclosed and shown herein may comprise a specific number of elements/components, the systems, devices and assemblies could be modified to include additional or fewer of such elements/components. For example, while any of the elements/components disclosed may be referenced as being singular, the embodiments disclosed herein could be modified to include a plurality of such elements/components. Selected features from one or more of the above-described embodiments may be combined to create alternative embodiments not explicitly described. All values and sub-ranges within disclosed ranges are also disclosed. The subject matter described herein intends to cover and embrace all suitable changes in technology.

What is claimed is:

1. A method for responsively adapting a user experience provided by an electronic device comprising:
receiving data about a first set of user interactions with the electronic device;
receiving data about a further set of user interactions with the electronic device;
detecting a change in the user's cognitive ability based on the data for the first set of user interactions and the data for the further set of user interactions; and
adapting the user experience provided by the electronic device in response to the detected change.

2. The method of claim 1 wherein the first set of user interactions and further set of user interactions with the electronic device are both performed for a purpose other than only to detect the change in the user's cognitive ability.

3. The method of claim 2 wherein the first set of user interactions and further set of user interactions are interactions that occur through one or more of: an event scheduling user interface; a user interface for accessing stored photos of the user; an electronic messaging user interface; and a user information user interface.

4. The method of claim 1 wherein the method is performed at one or more servers that communicate with the electronic device through a communication network.

5. The method of claim 1 wherein detecting the change in the user's cognitive ability comprises detecting a threshold change in a quantity of interactions occurring in the further set of user interactions compared to the first set of user interactions.

6. The method of claim 1 wherein detecting the change in the user's cognitive ability comprises detecting a threshold change in one or more of: a number of complex words input in the further set of user interactions compared to the first set of user interactions; word specificity occurring in the further set of user interactions compared to the first set of user interactions; a number of repeated words occurring in the further set of user interactions compared to the first set of user interactions; spelling accuracy of words input in the further set of user interactions compared to the first set of user interactions; a number of word classes included the further set of user interactions compared to the first set of user interactions; syntactic complexity included in user inputs in the further set of user interactions compared to the first set of user interactions; and text sentiment of user inputs in the further set of user interactions compared to the first set of user interactions.

7. The method of claim 1, wherein detecting the change in the user's cognitive ability comprises detecting a threshold change in appropriateness of diction and/or word order in user inputs occurring in the further set of user interactions compared to the first set of user interactions.

8. The method of claim 1, wherein detecting the change in the user's cognitive ability comprises detecting a threshold change in an accuracy of location-specific interactions with the electronic device in the further set of user interactions compared to the first set of user interactions.

9. The method of claim 1, wherein adaptively modifying the user experience provided by the electronic device comprises a change to a user interface including modifying one or more of: image sizes, font sizes, content complexity, white space around elements and/or text, size and visual affordance of interface elements, size of touch targets, number of displayed elements, abstractness of images, navigation bar size and availability, availability of delete/edit functions, availability of audio output.

10. A system, comprising:
a processor;
a memory coupled to the processor, the memory storing executable instructions that, when executed by the processor, cause the system to:
receive data about a first set of user interactions with an electronic device;
receive data about a further set of user interactions with the electronic device;
detect a change in the user's cognitive ability based on the data for the first set of user interactions and the data for the further set of user interactions; and
adapt a user experience in response to the detected change.

11. The system of claim 10 wherein the first set of user interactions and further set of user interactions are both performed with the electronic device for a purpose other than only to detect the change in the user's cognitive ability.

12. The system of claim 11 wherein the first set of user interactions and further set of user interactions are interactions that occur through one or more of: an event scheduling user interface; a user interface for accessing stored photos of the user; an electronic messaging user interface; and a user information user interface.

13. The system of claim 10 wherein the system is a server that communicates with the electronic device through a communication network.

14. The system of claim 10 wherein the system detects the change in the user's cognitive ability by detecting a threshold change in a quantity of interactions occurring in the further set of user interactions compared to the first set of user interactions.

15. The system of claim 10 wherein the system detects the change in the user's cognitive ability based on detecting a threshold change in one or more of: a number of complex words input in the further set of user interactions compared to the first set of user interactions; word specificity occurring in the further set of user interactions compared to the first set of user interactions; a number of repeated words occurring in the further set of user interactions compared to the first set of user interactions; spelling accuracy of words input in the further set of user interactions compared to the first set of user interactions; a number of word classes included the further set of user interactions compared to the first set of user interactions; syntactic complexity included in user inputs in the further set of user interactions compared to the first set of user interactions; and text sentiment of user inputs in the further set of user interactions compared to the first set of user interactions.

16. The system of claim 10, wherein the system detects the change in the user's cognitive ability based on detecting a threshold change in appropriateness of diction and/or word order in user inputs occurring in the further set of user interactions compared to the first set of user interactions.

17. The system of claim 10, wherein the system detects the change in the user's cognitive ability based on detecting a threshold change in an accuracy of location-specific interactions with the electronic device in the further set of user interactions compared to the first set of user interactions.

18. The system of claim 10, wherein the system adaptively modifies the user experience provided by the electronic device by causing a change to a user interface including modifying one or more of: image sizes, font sizes, content complexity, white space around elements and/or text, size and visual affordance of interface elements, size of touch targets, number of displayed elements, abstractness of images, navigation bar size and availability, availability of delete/edit functions, availability of audio output.

19. A method for responsively adapting a user experience provided by an electronic device comprising:
receiving data about user interactions with the electronic device;
adapting the user experience provided by the electronic device in response to detecting, based on the data about user interactions, that a user's cognitive ability is at a level that does not correspond to the user experience.

20. The method of claim 19 wherein the user interactions are performed with the electronic device for a purpose other than only to detect the user's cognitive ability.
